(19) United States
(12) Patent Application Publication
(10) Pub. No.: US 2017/0188824 A1
(43) Pub. Date: Jul. 6, 2017

(54) OPTICAL MEASUREMENT APPARATUS FOR EYEBALL

(71) Applicant: FUJI XEROX CO., LTD., Tokyo (JP)

(72) Inventors: Kazuyuki MATSUSHITA, Ebina-shi (JP); Kazutaka TAKEDA, Ebina-shi (JP); Kohei YUKAWA, Ebina-shi (JP)

(73) Assignee: FUJI XEROX CO., LTD., Tokyo (JP)

(21) Appl. No.: 15/463,124

(22) Filed: Mar. 20, 2017

Related U.S. Application Data
(63) Continuation of application No. PCT/JP2015/082595, filed on Nov. 19, 2015.

(30) Foreign Application Priority Data
Nov. 26, 2014 (JP) 2014-239091

(51) Int. Cl. A61B 3/117; A61B 5/145; A61B 5/1455; A61B 3/00

(52) U.S. Cl. CPC A61B 3/117; A61B 3/0025; A61B 5/14532; A61B 5/14551

(57) ABSTRACT

An optical measurement apparatus for an eyeball, including: a light irradiation section that is disposed on an inner canthus side of the eyeball and that includes a light source, a polarization portion that performs polarization control of light from the light source, and a light reflection portion that is disposed closer to an inward side of an eyeball than the polarization portion is and that reflects light subjected to the polarization control by the polarization portion such that the light travels across an anterior chamber of the eyeball; and a light reception section that is disposed on an outer canthus side of the eyeball, that receives the light traveling across the anterior chamber without further going through a light reflection portion that changes a polarization state of light, and that detects a change of the polarization state of the received light.

### CROSS REFERENCE TO RELATED APPLICATION

This is a continuation of International Application No. PCT/JP2015/082595 filed on Nov. 19, 2015, and claims priority from Japanese Patent Application No. 2014-239091, filed on Nov. 26, 2014.

### BACKGROUND

### Technical Field

The present invention relates to an optical measurement apparatus for an eyeball.

### SUMMARY

According to an aspect of the invention, there is provided an optical measurement apparatus for an eyeball, including: a light irradiation section that is disposed on an inner canthus side of the eyeball and that includes a light source, a polarization portion that performs polarization control of light from the light source, and a light reflection portion that is disposed closer to an inward side of an eyeball than the polarization portion is and that reflects light subjected to the polarization control by the polarization portion such that the light travels across an anterior chamber of the eyeball; and a light reception section that is disposed on an outer canthus side of the eyeball, receives the light traveling across the anterior chamber without further going through a light reflection portion that changes a polarization state of light, and detects a change of the polarization state of the received light.

### BRIEF DESCRIPTION OF DRAWINGS

Exemplary embodiment(s) of the present invention will be described in detail based on the following figures, wherein:

FIG. 1 is a view illustrating an example of a configuration of an optical measurement apparatus in which a first exemplary embodiment is applied;

FIG. 2 is a perspective view of the optical measurement apparatus viewed from a back side (inward side);

FIG. 3 is a view describing a relationship between an eyeball and an optical path in an optical system;

FIG. 4 is a view describing a method of measuring a rotation angle (optical rotation degree) of a vibration surface caused by an optically active substance contained in aqueous humor in an anterior chamber, by using the optical measurement apparatus;

FIGS. 5A and 5B are views describing an influence of a mirror in a light emission system. Here, FIG. 5A illustrates a case where light does not pass through the anterior chamber so as to travel across the anterior chamber, and FIG. 5B illustrates a case where light passes through the anterior chamber so as to travel across the anterior chamber;

FIGS. 6A and 6B are views describing a method of measuring the angle of the mirror. FIG. 6A illustrates the method of measuring the angle of the mirror by using a stepping motor included in an adjustment section, and FIG. 6B illustrates the method of measuring the angle of the mirror through a mirror angle measurement section including a light source which emits beam-like measurement light toward the mirror, and an image pickup device;

FIGS. 7A, 7B, and 7C are views describing an axis of rotation when the angle of the mirror is changed. Here, FIG. 7A illustrates a case where the axis of rotation coincides with a reflection point on the mirror, FIG. 7B illustrates a case where the axis of rotation coincides with the center of the mirror, and FIG. 7C illustrates a case where the axis of rotation coincides with an end on a back side (inward side) in a forward/backward direction of the mirror;

FIGS. 8A and 8B are views describing a case where the mirror is not used and a case where the mirror is used in a light reception system. Here, FIG. 8A illustrates a case where the mirror is not used in the light reception system, and FIG. 8B illustrates a case where the mirror is used in the light reception system;

FIGS. 9A and 9B are views describing the light emission system in the optical system of an optical measurement apparatus for an eyeball, in which a second exemplary embodiment is applied. Here, FIG. 9A illustrates a case where the optical path does not pass through the anterior chamber so as to travel across the anterior chamber, and FIG. 9B illustrates a case where the optical path passes through the anterior chamber so as to travel across the anterior chamber; and

FIGS. 10A and 10B are views describing the light emission system in the optical system of an optical measurement apparatus for an eyeball, in which a third exemplary embodiment is applied. Here, FIG. 10A illustrates a case where the optical path does not pass through the anterior chamber so as to travel across the anterior chamber, and FIG. 10B illustrates a case where the optical path passes through the anterior chamber so as to travel across the anterior chamber.

### DETAILED DESCRIPTION

Hereinafter, with reference to the accompanying drawings, exemplary embodiments of the present invention will be described. In the accompanying drawings, in order to make a relationship between an eyeball and an optical path clear, the eyeball is large-scaled compared to other members (such as an optical system to be described later).

### First Exemplary Embodiment

### Optical Measurement Apparatus 1

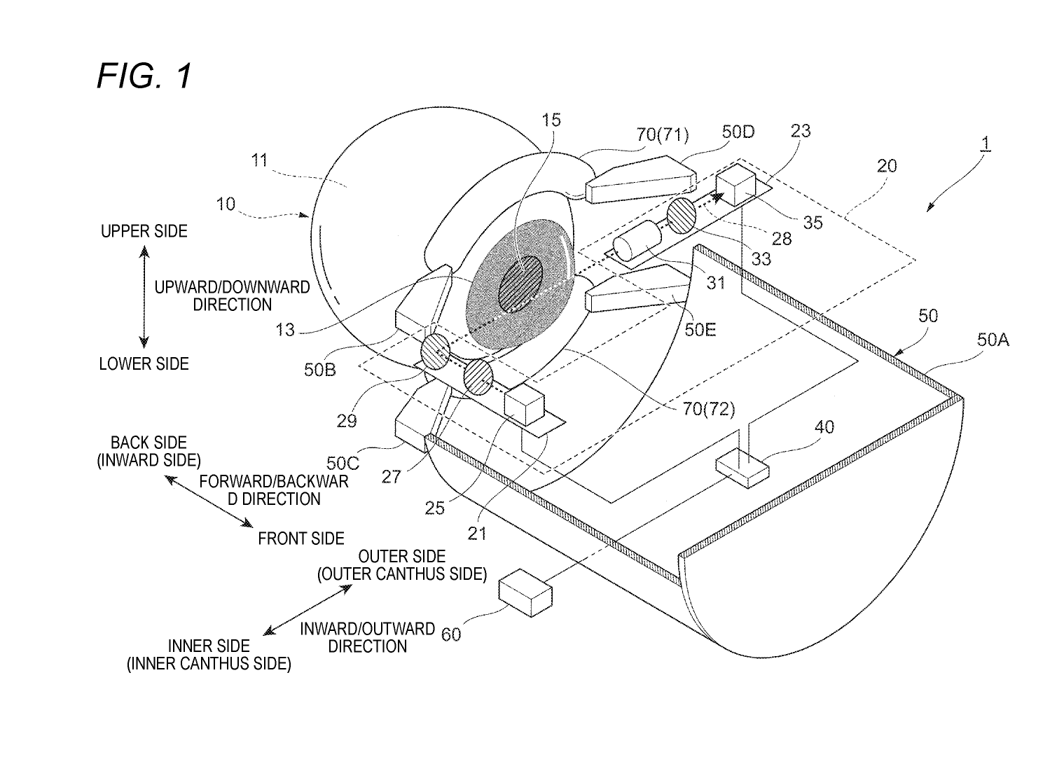

FIG. 1 is a view illustrating an example of a configuration of an optical measurement apparatus 1 in which a first exemplary embodiment is applied. An eyeball 10 illustrated in FIG. 1 is a left eye.

The optical measurement apparatus 1 includes an optical system 20 that is used in measurement related to aqueous humor inside an anterior chamber 13 (will be described later) of the eyeball 10 of a measurement subject (testee), a control section 40 that controls the optical system 20, a holding section 50 that holds the optical system 20 and the control section 40, a calculation section 60 that calculates characteristics of the aqueous humor based on data measured by using the optical system 20, and an eyelid pressing section 70 that comes into contact with an eyelid of the measurement subject and presses the eyelid.

In the description below, a direction crossing the upper side of the sheet and the lower side of the sheet regarding the optical measurement apparatus 1 illustrated in in FIG. 1 is sometimes referred to as upward/downward direction. In addition, a direction crossing the front side of the measurement subject and the hack side (inward side) of the measurement subject illustrated in FIG. 1 is sometimes referred to as forward/backward direction. In addition, a direction crossing the inner side (inner canthus side, nose side) and the outer side (outer canthus side, ear side) when viewed from the measurement subject of the optical measurement apparatus 1 illustrated in FIG. 1 is sometimes referred to as inward/outward direction.

In addition, the characteristics of the aqueous humor measured by the optical measurement apparatus 1 in which the first exemplary embodiment is applied denotes a rotation angle (optical rotation degree α_{M}) of a vibration surface of linearly polarized light caused by an optically active substance contained in the aqueous humor, a color absorbance degree (circular dichroism) with respect to circularly polarized light, and the like. The vibration surface of linearly polarized light denotes a surface where the electric field of the linearly polarized light vibrates.

The optical system 20 includes a light emission system 21 that emits light to the anterior chamber 13 (will be described later) of the eyeball 10, and a light reception system 23 that receives light which has passed through the anterior chamber 13.

First, the light emission system 21 as an example of a light irradiation section includes a light emission portion 25, a polarizer 27, and a mirror 29.

The light emission portion 25 as an example of a light source may be a light source having a wide wavelength width, such as a light emitting diode (LED) and a lamp or may be a light source having a narrow wavelength width such as a laser. Otherwise, the light emission portion 25 may include plural LEDs, lamps, or lasers. As described below, it is preferable to be able to use plural wavelengths.

The polarizer 27 as an example of a polarization portion is a Nicol prism, for example. From rays of incident light, the polarizer 27 allows linearly polarized light having a predetermined vibration surface to pass through.

The mirror 29 as an example of a light reflection portion reflects light which has passed through the polarizer 27 such that an optical path 28 indicated with the dotted line is refracted.

Subsequently, the light reception system 23 as an example of a light reception section includes a compensator 31, an analyzer 33, and a light reception portion 35. That is, in the light reception system 23, a mirror for refracting the optical path 28 is not used.

For example, the compensator 31 is a magneto-optic element such as a Faraday element in which a garnet or the like is used. The compensator 31 rotates the vibration surface of linearly polarized light in response to a magnetic field.

The analyzer 33 is a member similar to the polarizer 27 and allows linearly polarized light having the predetermined vibration surface to pass through.

The light reception portion 35 is a light receiving element such as a silicon diode and outputs an output signal corresponding to the intensity of light.

The control section 40 controls the light emission portion 25, the compensator 31, the light reception portion 35, and the like in the optical system 20, thereby obtaining measurement data related to the characteristics of the aqueous humor.

The holding section 50 is an approximately cylindrical housing which holds the optical system 20 and the control section 40. The holding section 50 illustrated in FIG. 1 exhibits a shape realized by cutting a cylinder along a plane parallel to an axial direction such that the optical system 20 is easily recognized. In addition, the shape of the holding section 50 may be a different shape. For example, a cross section of the holding section 50 may have a quadrangular or elliptic tube shape. The holding section 50 will be described later in detail.

The calculation section 60 receives the measurement data from the control section 40 and calculates the characteristics of the aqueous humor.

The eyelid pressing section 70 is provided in the holding section 50 and presses eyelids (upper eyelid and lower eyelid) by being in contact with the eyelids, thereby maintaining the eyelids in an open state. The eyelid pressing section 70 includes an upper eyelid pressing section 71 and a lower eyelid pressing section 72.

The optical measurement apparatus I may not include the eyelid pressing section 70.

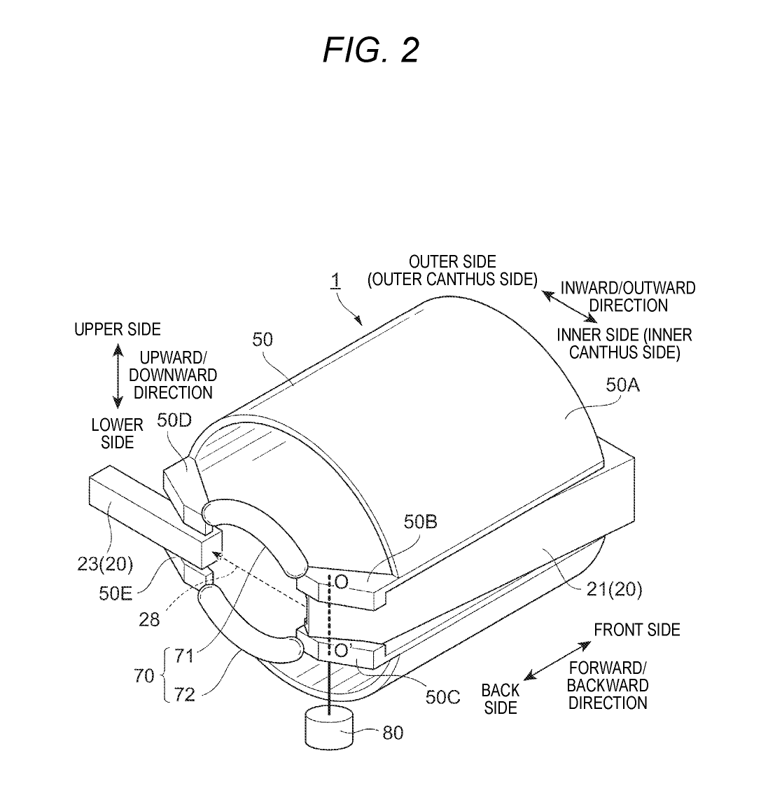

FIG. 2 is a perspective view of the optical measurement apparatus 1 viewed from a back side (inward side). Illustration of the calculation section 60 is omitted.

Here, the holding section 50 will be described.

The holding section 50 includes a cylindrical main body 50A, and support portions 50B, 50C, 50D, and 50E. The support portions 50B, 50C, 50D, and 50E are provided by being fixed to an end portion of the main body 50A on the back side (inward side). The support portions 50B and 50C support the light emission system 21 and respectively support one end portion of the upper eyelid pressing section 71 and one end portion of the lower eyelid pressing section 72. The support portions 50D and 50E support the light reception system 23 and respectively support the other end portion of the upper eyelid pressing section 71 and the other end portion of the lower eyelid pressing section 72.

The support portions 50B and 50C supporting the light emission system 21 are provided with an axis O-O′ utilized when the direction of light emitted from the light emission system 21 is changed. As described below, while having the axis O-O′ as the center, when the mirror 29 or the light emission system 21 in the light emission system 21 is rotated (moved) (the angle thereof is changed), the direction of light emitted from the light emission system 21 is changed.

Moreover, the optical measurement apparatus I includes an adjustment section 80 that can adjust the direction of light by rotating (moving) (changing the angle thereof) the mirror 29 or the light emission system 21 in the light emission system 21 while having the axis O-O′ as the center.

The adjustment section 80 may include a motor or the like so as to adjust the direction of light by rotating the mirror 29 or the light emission system 21 in the light emission system 21, based on the controlling of the control section 40. In addition, the adjustment section 80 may include a mechanism such as a rotatable dial such that the measurement subject adjusts the direction of light by manually rotating the mirror 29 or the light emission system 21 in the light emission system 21. That is, the adjustment section 80 may have a different mechanism as long as the mechanism can adjust the angle of the mirror 29 in the light emission system 21.

In a case where the optical measurement apparatus 1 does not include the eyelid pressing section 70, the support portions 50B and 50C are configured to support the light emission system 21, and the support portions 50D and 50E are configured to support the light reception system 23.

### Relationship Between Eyeball 10 and Optical Path 28 in Optical System 20

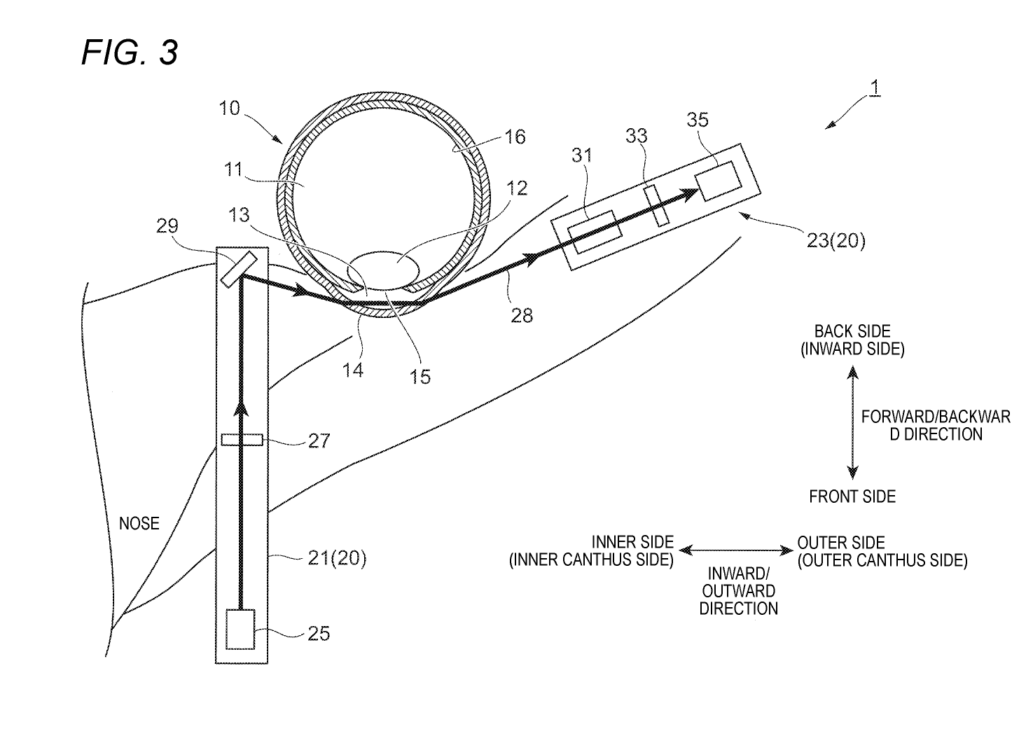

FIG. 3 is a view describing a relationship between the eyeball 10 and the optical path 28 in the optical system 20. FIG. 3 illustrates a state where a person (measurement subject) is viewed from the head side (upper side). In addition, in the view, a part of the optical system 20 appears to be embedded inside the face in relation to the uneven shape of the surface of the face. Actually, the optical system 20 is disposed on the surface of the face.

Subsequently, with reference to FIG. 3, a relationship between the eyeball 10 and the optical path 28 of the optical system 20 will be described.

Here, first, the structure of the eyeball 10 will be described. Subsequently, a relationship between the eyeball 10 and the optical path 28 of the optical system 20 will be described in detail.

As illustrated in FIG. 3, the eyeball 10 has a substantially spherical outer shape and a glass body 11 is present at the center. A crystalline lens 12 playing a role as a lens is embedded in a part of the glass body 11. The anterior chamber 13 is present on the front side of the crystalline lens 12, and a cornea 14 is present on the front side thereof. The anterior chamber 13 and the cornea 14 bulge out from the spherical shape in a convex shape.

The peripheral portion of the crystalline lens 12 is surrounded by the iris, and the center thereof is a pupil 15. Excluding a portion being in contact with the crystalline lens 12, the glass body 11 is covered with a retina 16.

The anterior chamber 13 is a region surrounded by the cornea 14 and the crystalline lens 12. The anterior chamber 13 has a circular shape when viewed from the front (refer to FIG. 1). The anterior chamber 13 is filled with the aqueous humor.

Subsequently, a positional relationship between the eyeball 10 and the optical path 28 of the optical system 20 will be described.

As illustrated in FIG. 3, in the optical system 20, light used for measuring the characteristics of the aqueous humor is emitted from the light emission portion 25 and travels forward along the optical path 28, thereby being incident on the light reception portion 35. That is, light emitted from the light emission portion 25 passes through the polarizer 27. Thereafter, the light is refracted by the mirror 29 in a direction of traveling across the anterior chamber 13 (direction parallel to the eye). The light passes through the anterior chamber 13 so as to travel across (inward/outward direction) the anterior chamber 13. Moreover, the light which has passed through the anterior chamber 13 is incident on the light reception portion 35 via the compensator 31 and the analyzer 33.

Here, as illustrated in FIG. 3, the light emitted from the light emission system 21 is incident on the anterior chamber 13 in an orientation toward the outer side (outer canthus side) in the inward/outward direction and in an orientation toward the front side in the forward/backward direction. In addition, the light which has passed through the anterior chamber 13 is incident on the light reception system 23 in the orientation toward the outer side (outer canthus side) in the inward./outward direction and in the orientation toward the back side (inward side) in the forward/backward direction.

That is, the light emission system 21 (mirror 29) is disposed such that the light emitted toward the anterior chamber 13 by the light emission system 21 obliquely travels toward the front side in the forward/backward direction. That is, the mirror 29 is disposed on the back side (inward side) with respect to an exposed portion (anterior chamber 13) of the eyeball 10 closer than the front side apex thereof.

In addition, the light reception system 23 is disposed so as to receive light obliquely traveling from the anterior chamber 13 toward the back side (inward side) in the forward/backward direction.

The disposition is performed due to the following reason. That is, light emitted from the light emission portion 25 passes through the cornea 14 and is incident on the anterior chamber 13. In this case, the light is refracted due to the anterior chamber 13 and the cornea 14 bulging out from the eyeball 10 in a convex shape, and due to the refractive index differences between air (refractive index: 1) and the cornea 14 (refractive index: 1.37 to 1.38), and the cornea 14 (refractive index: 1.37 to 1.38) and the aqueous humor (refractive index: approximately 1.34). That is, the optical path 28 is refracted toward the back side (inward, the eyeball 10 side) when light is incident on the cornea 14 and the anterior chamber 13 (aqueous humor), and the optical path 28 is further refracted toward the back side (inward side) when light is emitted from the anterior chamber 13 (aqueous humor) and the cornea 14. The light emission system 21 and the light reception system 23 are disposed in consideration of light passing through the cornea 14 and the anterior chamber 13 and being refracted toward the back side (inward side).

In addition, the nose (bridge of the nose) is positioned around the eye (eyeball 10) in the face, and there is a small space for setting the optical system 20. Moreover, when light deviates from the anterior chamber 13, accurate measurements cannot be performed. Thus, it is preferable to set the optical path 28 such that light does not deviate from the anterior chamber 13 and the optical path 28 passes through the anterior chamber 13 so as to travel across the anterior chamber 13.

In the illustrated optical measurement apparatus 1, the optical path 28 is set such that light is incident at an angle nearly parallel to the eyeball 10 and the optical path 28 travels across the anterior chamber 13. Therefore, in the light emission system 21, the space is intended to be effectively utilized by providing the mirror 29 and refracting the optical path 28 on the inner side (inner canthus side).

Meanwhile, in the light reception system 23, no mirror is provided and the optical path 28 is not refracted. The reason is that since there is nothing such as the nose limiting the space on the outer side (outer canthus side), a long light reception system 23 can be installed.

That is, in the first exemplary embodiment, the light emission system 21 is provided on the inner side (inner canthus side) and the light reception system 23 is disposed on the outer side (outer canthus side).

The optical path 28 is not limited to the illustrated configuration and is favorable as long as the optical path 28 is set such that light emitted from the light emission system 21 passes through the anterior chamber 13 so as to travel across the anterior chamber 13 and is received by the light reception portion 35. In addition, the circumstances where light passes through the anterior chamber 13 so as to travel across the anterior chamber 13 denote that the light passes through the anterior chamber 13 at an angle (that is, a range less than ±45 degrees with respect to a horizontal axis in the inward/outward direction) closer to the inward/outward direction than the upward/downward direction in a case where the eyeball 10 is viewed from the front, including a case where the light obliquely passes through the anterior chamber 13 in the forward/backward direction.

### Optical Measurement of Aqueous Humor

Subsequently, an example of calculating a glucose concentration of the aqueous humor in the anterior chamber 13 by using the optical measurement apparatus 1 will be described.

### Background of Measuring Glucose Concentration of Aqueous Humor

First, the background of measuring the glucose concentration of the aqueous humor will be described.

Self-blood glucose measurement is recommended for a type-1 diabetic patient and a type-2 diabetic patient (measurement subject) requiring insulin therapy. In self blood glucose measurement, in order to exactly control the blood glucose, the measurement subject measures his/her own blood glucose level by himself/herself at home or the like.

In a self-blood glucose measurement instrument currently on the market, a fingertip or the like is punctured with an injection needle and a very small quantity of blood is gathered, thereby measuring the glucose concentration in the blood. The self-blood glucose measurement is often recommended to be performed after each meal, before bed, or the like and is required to be performed once to several times a day. Particularly, in intensive insulin therapy, much more times of measurement are required.

Therefore, an invasive-type blood glucose level measurement method using a puncture-type self-blood glucose measurement instrument is likely to cause degradation of incentives with respect to the self-blood glucose measurement of the measurement subject due to distress from the pain when blood is gathered (during blood collection). Therefore, there are cases where it is difficult to efficiently conduct diabetic therapy.

Therefore, in place of the invasive-type blood glucose level measurement method such as puncturing, development of a noninvasive-type blood glucose level measurement method requiring no puncturing is carried out.

As the noninvasive-type blood glucose level measurement method, near infrared spectroscopy, optoacoustic spectroscopy, a method of utilizing optical activities, and the like are reviewed. In these methods, the blood glucose level is presumed from the glucose concentration.

In the near infrared spectroscopy or the optoacoustic spectroscopy, optical absorption spectrums or acoustic vibrations in blood inside a blood vessel of a finger are detected. However, in blood, cell substances such as red blood corpuscles and white blood corpuscles are present. Therefore, the methods are greatly influenced by light scattering. Moreover, in addition to blood inside a blood vessel, the methods are also influenced by the peripheral tissue. Thus, in these methods, a signal related to the glucose concentration is required to be detected from signals associated with an enormous number of substances such as protein and amino acid, and it is difficult to separate the signal therefrom.

Meanwhile, the aqueous humor in the anterior chamber 13 is substantially the same component as blood serums and includes protein, glucose, ascorbic acid, and the like. However, the aqueous humor is different from blood and does not include the cell substances such as red blood corpuscles and white blood corpuscles, thereby being less influenced by the light scattering. Thus, the aqueous humor is suitable for an optical measurement of the glucose concentration.

Protein, glucose, ascorbic acid, and the like contained in the aqueous humor are the optically active substances and have optical activities.

The optical measurement apparatus 1 in which the first exemplary embodiment is applied optically measures the concentration of the optically active substances containing glucose, from the aqueous humor by utilizing the optical activities.

Since the aqueous humor is a tissue fluid for transporting glucose, the glucose concentration of the aqueous humor is considered to be correlated to the glucose concentration in blood. According to a report regarding a measurement in which a rabbit is used, the time taken for transporting glucose from blood to the aqueous humor (transportation delay time) is within 10 minutes.

### Setting of Optical Path

In a technique of optically measuring the concentration of the optically active substances such as glucose contained in the aqueous humor, two optical paths can be set as follows.

In one optical path being different from the first exemplary embodiment illustrated in FIG. 3, light is incident at an angle nearly perpendicular to the eyeball 10, that is, along the forward/backward direction, the light is reflected by the interface between the cornea 14 and the aqueous humor or the interface between the aqueous humor and the crystalline lens 12, and the reflected light is received (detected). In the other optical path as in the first exemplary embodiment illustrated in FIG. 2, light is incident at an angle intersecting the forward/backward direction, specifically at an angle nearly parallel to the eyeball 10, and the light which has passed through the anterior chamber 13 so as to travel across the anterior chamber 13 is received (detected).

In an optical path such as the former above in which light is incident at an angle nearly perpendicular to the eyeball 10, there is a possibility that the light reaches the retina 16. Particularly, in a case of using a laser having high coherency in the light emission portion 25, it is not preferable when light reaches the retina 16.

In contrast, in an optical path such as that in the first exemplary embodiment, that is, the latter above in which light is incident at an angle nearly parallel to the eyeball 10, the light passes through the anterior chamber 13 so as to travel across the anterior chamber 13 via the cornea 14, and the light which has passed through the aqueous humor is received (detected). Therefore, the light is restrained from reaching the retina 16.

The rotation angle (optical rotation degree) of the vibration surface caused by the optically active substance depends on the optical path length. As the optical path length is elongated, the optical rotation degree increases. Thus, a long optical path length can be set by causing light to pass through the anterior chamber 13 so as to travel across the anterior chamber 13.

### Calculation of Concentration of Optically Active Substance

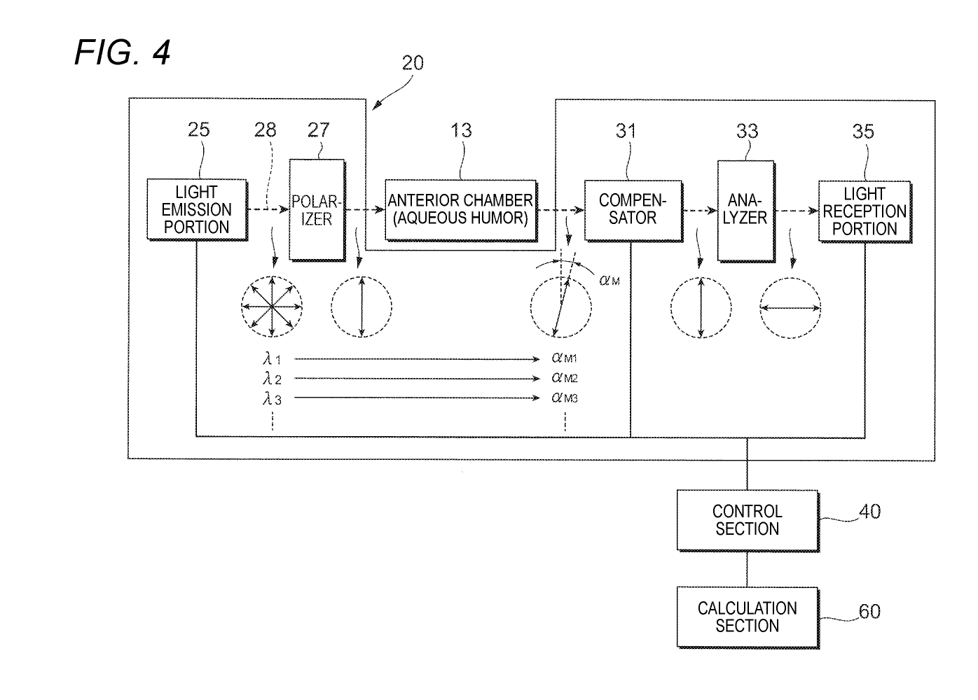

FIG. 4 is a view describing a method of measuring the rotation angle (optical rotation degree) of the vibration surface caused by the optically active substance contained in the aqueous humor in the anterior chamber 13, by using the optical measurement apparatus 1. Here, in order to make description easy, the optical path 28 is configured not to be refracted and illustration of the mirror 29 is omitted.

In addition, in each of the spaces among the light emission portion 25, the polarizer 27, the anterior chamber 13, the compensator 31, the analyzer 33, and the light reception portion 35 illustrated in FIG. 4, the states of polarized light viewed in traveling directions of the light are respectively indicated with arrows in a circle.

The light emission portion 25 emits light having a random vibration surface. The polarizer 27 allows linearly polarized light having the predetermined vibration surface to pass through. In FIG. 4, as an example, linearly polarized light having the vibration surface parallel to the sheet passes through.

The vibration surface of the linearly polarized light which has passed through the polarizer 27 is rotated by the optically active substance contained in the aqueous humor in the anterior chamber 13. In FIG. 4, the vibration surface rotates by the angle α_{M} (optical rotation degree α_{M}).

Subsequently, the vibration surface which has been rotated by the optically active substance contained in the aqueous humor in the anterior chamber 13 is returned to the original state by the compensator 31. In a case where the compensator 31 is a magneto-optic element such as a Faraday element, a magnetic field is applied to the compensator 31 and the vibration surface of light passing through the compensator 31 is rotated.

The linearly polarized light which has passed through the analyzer 33 is received by the light reception portion 35 and is converted into an output signal corresponding to the intensity of light.

Here, an example of the method of measuring the optical rotation degree α_{M} by using the optical system 20 will be described.

First, in a state where light emitted from the light emission portion 25 is prohibited from passing through the anterior chamber 13, while the optical system 20 including the light emission portion 25, the polarizer 27, the compensator 31, the analyzer 33, and the light reception portion 35 is used, the compensator 31 and the analyzer 33 are set such that an output signal from the light reception portion 35 is minimized. In the example illustrated in FIG. 4, in a state where light is prohibited from passing through the anterior chamber 13, the vibration surface of the linearly polarized light which has passed through the polarizer 27 becomes orthogonal to the vibration surface passing through the analyzer 33.

Subsequently, a state where light passes through the anterior chamber 13 is established. Then, the vibration surface of the light rotates due to the optically active substance contained in the aqueous humor in the anterior chamber 13. Therefore, the output signal from the light reception portion 35 deviates from the minimum value. The vibration surface is rotated by applying a magnetic field to the compensator 31 such that the output signal from the light reception portion 35 is minimized. That is, the vibration surface of the light emitted from the compensator 31 is caused to be orthogonal to the vibration surface passing through the analyzer 33.

The angle of the vibration surface rotated by the compensator 31 corresponds to the optical rotation degree α_{M} caused by the optically active substance contained in the aqueous humor. Here, the relationship between the magnitude of the magnetic field applied to the compensator 31 and the angle of the rotated vibration surface is known in advance. Therefore, based on the magnitude of the magnetic field applied to the compensator 31, the optical rotation degree α_{M} is ascertained.

Specifically, rays of light having plural wavelengths λ (wavelengths λ₁, λ₂, λ₃, and so on) are incident on the aqueous humor in the anterior chamber 13 from the light emission portion 25, and the optical rotation degrees α_{M} (optical rotation degrees α_{M1}, α_{M2}, α_{M3}, and so on) are respectively obtained with respect to the wavelengths. The sets of the wavelength λ and the optical rotation degree α_{M} are taken into the calculation section 60, and the concentration of an intended optically active substance is calculated.

The concentration of the optically active substance calculated by the calculation section 60 may be displayed through a display section (not illustrated) included in the optical measurement apparatus 1 or may be output to a different terminal device (not illustrated) such as a personal computer (PC) via an output section (not illustrated) included in the optical measurement apparatus 1.

In addition, as described above, the aqueous humor contains plural optically active substances. Thus, the measured optical rotation degree α_{M} is the sum of each of the optical rotation degrees α_{M} of the plural optically active substances. Therefore, the concentration of the intended optically active substance (here, glucose) is required to be calculated from the measured optical rotation degree α_{M}. For example, the concentration of the intended optically active substance can be calculated by using a known method such as that disclosed in JP-A-09-138231. Thus, description will be omitted herein.

In addition, in FIG. 4, both the vibration surface of the polarizer 27 and the vibration surface before passing through the analyzer 33 are parallel to the sheet. However, in a state where light emitted from the light emission portion 25 is prohibited from passing through the anterior chamber 13, in a case where the vibration surface is rotated by the compensator 31, the vibration surface before passing through the analyzer 33 may incline from a plane parallel to the sheet. That is, in a state where light is prohibited from passing the aqueous humor in the anterior chamber 13, the compensator 31 and the analyzer 33 are favorably set such that the output signal from the light reception portion 35 is minimized.

In addition, here, an example of using the compensator 31 is described as a method of obtaining the optical rotation degree α_{M}. However, the optical rotation degree α_{M} may be obtained by using a portion other than the compensator 31. Moreover, here, an orthogonal polarizer method (however, the compensator 31 is used) which is the most basic measurement method of measuring the rotation angle (optical rotation degree α_{M}) of the vibration surface is described. However, other measurement methods such as a rotation analyzer method, a Faraday modulation method, and an optical delay modulation method may be applied.

### Influence on Polarization State of Mirror 29

As described above, the nose (ridge of the nose) is positioned around the eye (eyeball 10) in the face, and there is a small space for setting the optical system 20. Therefore, in order to cause light to pass through the anterior chamber 13 so as to travel across the anterior chamber 13, it is preferable that the light emission system 21 is disposed on the inner canthus (nose) side and the optical path 28 is refracted by using the mirror 29. It is preferable that the light reception system 23 is disposed on the outer side (outer canthus side) and no mirror is used.

When measuring the concentration of an optically active substance such as glucose by applying optical activities, the optical rotation degree α_{M} is required to be measured as described above. The optical rotation degree α_{M} is rotation of the vibration surface of polarized light. Thus, when the vibration surface of polarized light rotates or the state of polarized light (polarization state) changes due to an influence other than optical activities caused by the optically active substance such as glucose in the aqueous humor, the measurement of the glucose concentration becomes inaccurate. That is, the accuracy of the measurement is degraded.

The factors rotating the vibration surface other than optical rotation caused by the optically active substance or changing the polarization state include reflection of the mirror 29 in the light emission system 21, and double refraction caused by the cornea 14.

### Light Emission System 21

First, the influence on the polarization state of the mirror 29 in the light emission system 21 will be described.

Generally, in reflection of a mirror, the reflectance of a component (P) parallel to the incident surface and the reflectance of a component (S) perpendicular to the incident surface depend on the refractive index and the incident angle of the mirror. Therefore, when polarized light is incident on the mirror, the polarization state of the reflected light is sometimes different (changed) from the polarization state of the incident light due to the incident angle. For example, in a case where linearly polarized light is incident, reflected light sometimes becomes linearly polarized light at a certain incident angle, and reflected light sometimes becomes elliptically polarized light at a different incident angle.

If the refractive index of a mirror, the polarization state of incident light (orientation of the vibration surface, and states of linearly polarized light, elliptically polarized light, and the like), and the incident angle are known, the polarization state of reflected light can be calculated.

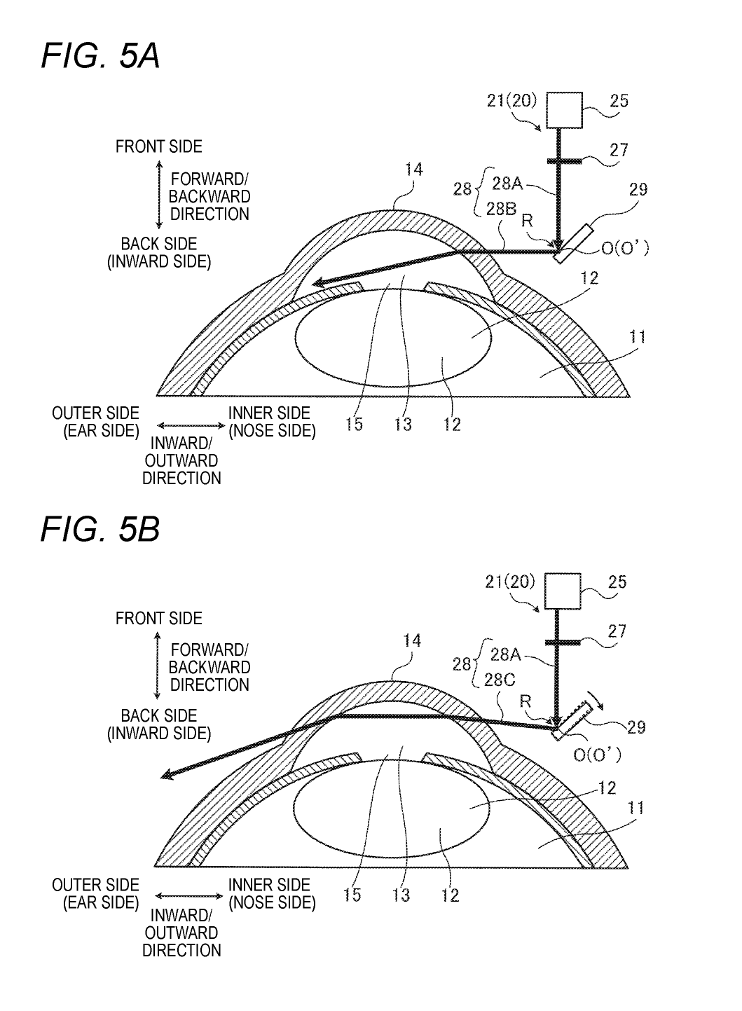

FIGS. 5A and 5B are views describing the influence of the mirror 29 in the light emission system 21. Here, FIG. 5A illustrates a case where light does not pass through the anterior chamber 13 so as to travel across the anterior chamber 13, and FIG. 5B illustrates a case where light passes through the anterior chamber 13 so as to travel across the anterior chamber 13.

As illustrated in FIG. 5A, incident light 28A which is emitted from the light emission portion 25 and is incident on the mirror 29 is reflected by the mirror 29 and is oriented toward the eyeball 10. However, reflected light 28B which is reflected by the mirror 29 does not pass through the anterior chamber 13 so as to travel across the anterior chamber 13 and is oriented toward the back side (inward, the eyeball 10 side).

As illustrated in FIG. 5B, the angle of the mirror 29 is changed such that reflected light 28C reflected by the mirror 29 is adjusted so as to pass through the anterior chamber 13 and to travel across the anterior chamber 13.

In FIG. 5B, the angle of the mirror 29 is changed without moving the light emission portion 25, and the reflected light 28B from the mirror 29 is changed into the reflected light 28C. In this case, the polarization states of the reflected light 28B and the reflected light 28C can be different from each other.

Therefore, even if the polarization state of the reflected light 28B from the mirror 29 in FIG. 5A is ascertained, since the angle of the mirror 29 is changed as illustrated in FIG. 5B, the polarization state of the reflected light 28C is no longer ascertained. Thus, even if light which has passed through the anterior chamber 13 so as to travel across the anterior chamber 13 is measured, the optical rotation degree α_{M} of the optically active substance contained in the aqueous humor cannot be accurately calculated.

However, if the angle of the mirror 29 in FIG. 5B is ascertained, the polarization state of the reflected light 28C can be calculated. Thus, in consideration of a change of the polarization state caused by the mirror 29, the optical rotation degree α_{M} of the optically active substance contained in the aqueous humor is more accurately calculated.

That is, in FIG. 5B, the angle of the mirror 29 is required to be measured.

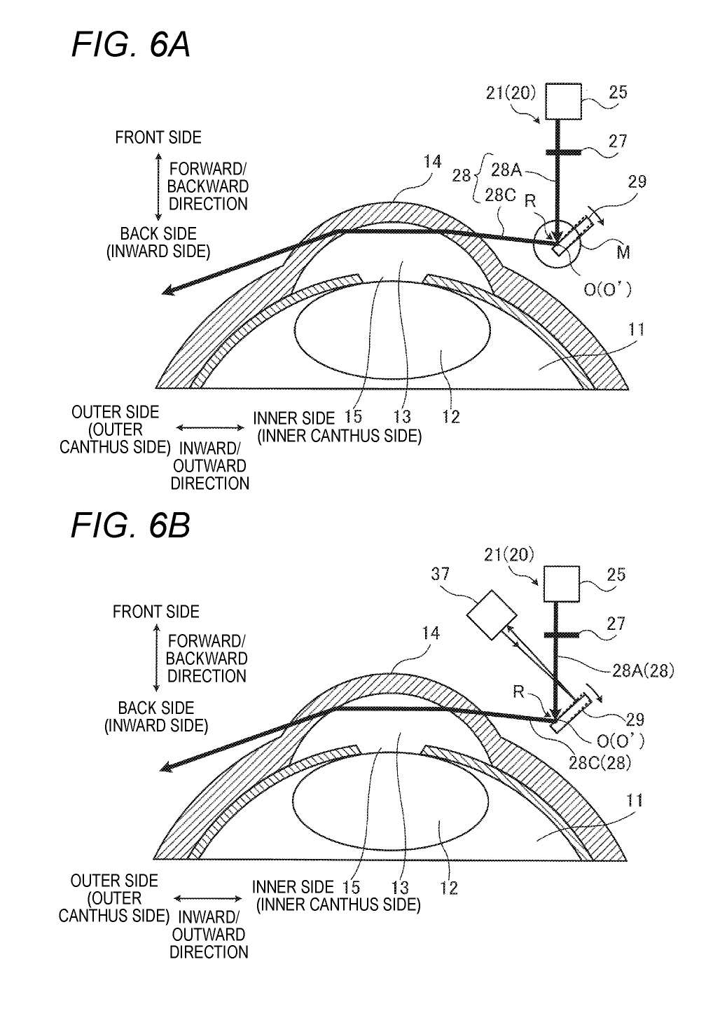

FIGS. 6A and 6B are views describing a method of measuring the angle of the mirror 29. FIG. 6A illustrates the method of measuring the angle of the mirror 29 by using a stepping motor M included in the adjustment section 80, and FIG. 6B illustrates the method of measuring the angle of the mirror 29 through a mirror angle measurement section 37 including a light source which emits beam-like measurement light toward the mirror 29, and an image pickup device.

First, the method of measuring the angle of the mirror 29 by using the stepping motor M illustrated in FIG. 6A will be described. The stepping motor M is an example of the adjustment section and is an example of the angle measurement section.

The stepping motor M is configured to include a rotor (magnet) and plural coils provided around the rotor. The plural coils are excited through a predetermined method, and the rotor of the stepping motor M rotates at a minute angle. That is, the rotation angle of the stepping motor M is set when a current exciting the coils is supplied.

While having the angle of the mirror 29 illustrated in FIG. 5A as the reference, the stepping motor M is rotated, thereby realizing the angle of the mirror 29 illustrated in FIG. 5B. In this case, a change of the angle of the mirror 29 is measured from the rotation angle of the stepping motor M. That is, the angle of the mirror 29 is ascertained. Thus, the polarization state of the reflected light 28C of the mirror 29 can be calculated.

The stepping motor M is controlled by the control section 40.

Subsequently, the method of measuring the angle of the mirror 29 by using the mirror angle measurement section 37 illustrated in FIG. 6B will be described. The mirror angle measurement section 37 is another example of the angle measurement section.

The mirror angle measurement section 37 includes the light source which emits beam-like measurement light toward the mirror 29, and the image pickup device which includes plural light reception cells receiving light reflected from the mirror 29.

The angle of the mirror 29 illustrated in FIG. 5A is applied as the reference. In this case, beam-like angle measurement light emitted from the light source is reflected by the surface of the mirror 29 and is incident on any one of the plural light reception cells of the image pickup device. The angle of the mirror 29 is changed, thereby realizing the angle of the mirror 29 illustrated in FIG. 5B. Then, the beam-like angle measurement light emitted from the light source is reflected by the surface of the mirror 29 and is incident on any different one of the plural light reception cells of the image pickup device. That is, due to a positional shift (misalignment) of the light reception cell receiving the angle measurement light reflected by the surface of the mirror 29, a change of the angle of the mirror 29 is measured. That is, the angle of the mirror 29 is ascertained. Thus, the polarization state of reflected light of the mirror 29 can be calculated.

The light source emitting beam-like measurement light toward the mirror 29 may be an LED or a laser. The image pickup device receiving the measurement light reflected by the surface of the mirror 29 may be a CCD or a CMOS sensor.

In this case, the angle of the mirror 29 may be set by rotating the motor included in the adjustment section 80 or may be manually set (adjusted) by the measurement subject using a dial or the like included in the adjustment section 80.

The mirror angle measurement section 37 may be controlled by the control section 40.

The angle of the mirror 29 may be measured through the method of using the stepping motor M described above or a method other than the method using the mirror angle measurement section 37.

### Axis O-O′ of Rotation of Mirror 29

Here, the axis O-O′ of rotation when the angle of the mirror 29 is changed will be described. The angle of the mirror 29 is changed when the adjustment section 80 moves the mirror 29 around the axis O-O′. Here, such circumstances are expressed that the mirror 29 rotates around the axis O-O′.

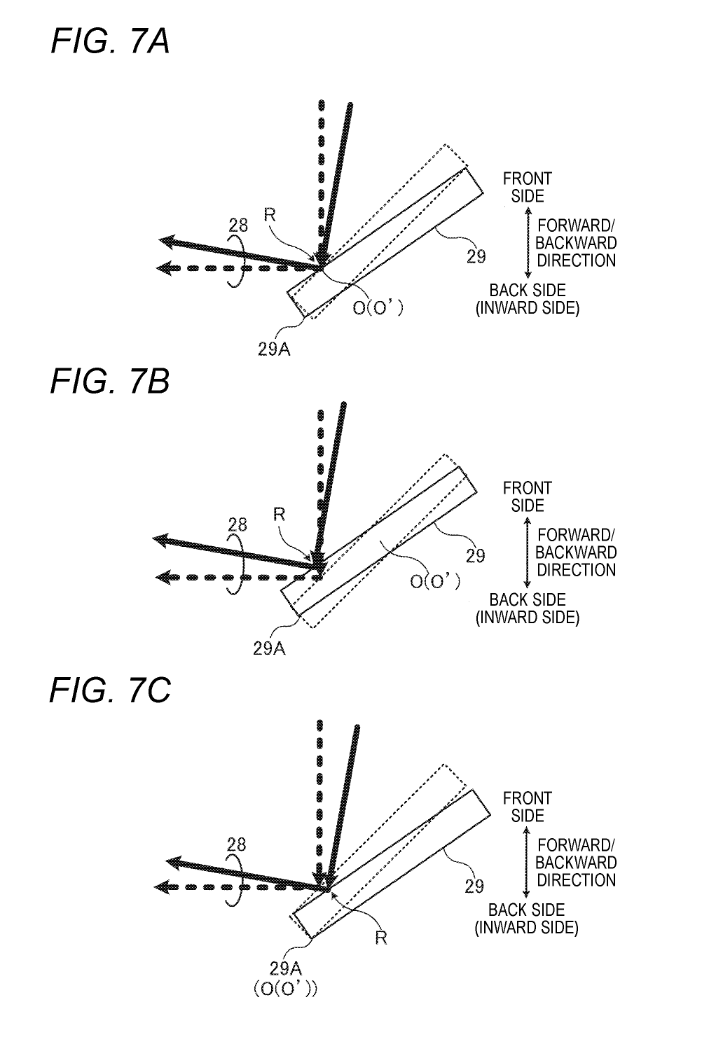

FIGS. 7A. 7B, and 7C are views describing the axis O-O′ of rotation (in the view, indicated as O (O′)) when the angle of the mirror 29 is changed. Here, FIG. 7A illustrates a case where the axis O-O′ of rotation coincides with a reflection point R on the mirror 29, FIG. 7B illustrates a case where the axis O-O′ of rotation coincides with the center of the mirror 29, and FIG. 7C illustrates a case where the axis O-O′ of rotation coincides with an end 29A on the back side (inward side) in the forward/backward direction of the mirror 29.

Here, the reflection point R of the optical path 28 in the mirror 29 is illustrated close to the back side (inward side) in the forward/backward direction of the mirror 29. In a case where the mirror 29 includes a member having a reflection surface, and a member which is on the back surface of the member having the reflection surface and supports the member having the reflection surface, the members as a whole are indicated as the mirror 29.

As illustrated in FIG. 7A, in a case where the axis O-O′ coincides with the reflection point R of the optical path 28 on the mirror 29, even if the angle of the mirror 29 is changed, the reflection point R does not move. Thus, the optical path 28 is easily adjusted.

As illustrated in FIG. 7B, in a case where the axis O-O′ and the reflection point R do not coincide with each other, such as a case where the axis O-O′ is on the center side of the mirror 29, when the angle of the mirror 29 is changed, the reflection point R of the optical path 28 on the mirror 29 moves. Thus, compared to a case where the axis O-O′ coincides with the reflection point R, it is difficult to adjust the optical path 28. As the axis O-O′ and the reflection point R are separated from each other in distance, the movement quantity increases. in addition, in a case of FIG. 7B, the end 29A of the mirror 29 moves. As illustrated in FIG. 3, the mirror 29 is provided near the eyeball 10 in the face. Thus, depending on the distance between the mirror 29 and the eyeball 10 in the face or the distance between the axis O-O′ and the reflection point R, there is a possibility that the end 29A of the mirror 29 moves and the mirror 29 hits the face (eyeball 10).

As illustrated in FIG. 7C, in a case where the axis O-O′ coincides with the end 29A of the mirror 29, when the angle of the mirror 29 is changed, the reflection point R in the optical path 28 on the mirror 29 moves. Thus, compared to a case where the axis O-O′ coincides with the reflection point R, it is difficult to adjust the optical path 28. However, since the end 29A of the mirror 29 does not move, the possibility that the minor 29 hits the face (eyeball 10) is reduced.

As described above, when the axis O-O′ for rotating the mirror 29 coincides with the reflection point R, the optical path 28 is easily adjusted. Meanwhile, when the axis O-O′ for rotating the mirror 29 coincides with the end 29A of the back side (face side) in the forward/backward direction of the mirror 29, the distance between the mirror 29 and the face is restrained from changing.

Thus, in order to prohibit the reflection point R from moving as much as possible, it is preferable to provide the axis O-O′ for rotating the mirror 29 at a position near the reflection point R in the region of the mirror 29 and it is more preferable that the axis O-O′ coincides with the reflection point R. In addition, in order to reduce the possibility that the mirror 29 hits the face (eyeball 10), it is preferable to provide the axis O-O′ in a region on a side close to the face side in the region of the mirror 29, and it is more preferable to provide the axis O-O′ at the end portion on a side close to the face side.

### Light Reception System 23

Subsequently, a reason not using the mirror in the light reception system 23 will be described.

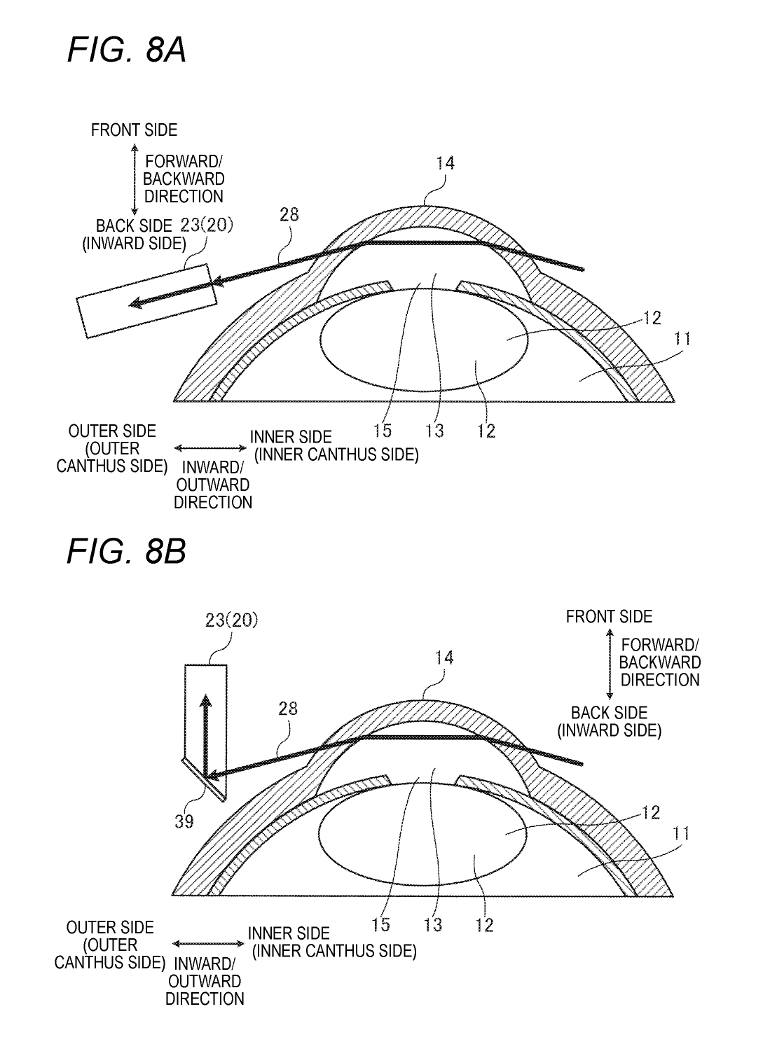

FIGS. 8A and 8B are views describing a case where the mirror is not used and a case where the mirror is used in the light reception system 23. Here, FIG. 8A illustrates a case where the mirror is not used the light reception system 23, and FIG. 8B illustrates a case where a mirror 39 is used in the light reception system 23. In the light reception system 23, illustrations of the compensator 31, the analyzer 33, and the light reception portion 35 are omitted.

As illustrated in FIG. 8A, in a case where the mirror is not used in the light reception system 23, light traveling across the anterior chamber 13 of the eyeball 10 passes through the cornea 14 and is incident on the light reception system 23 in which the mirror is not used. For example, the light is incident on the compensator 31 illustrated in FIG. 3. In this case, no change of the polarization state caused due to reflection of the mirror is received.

Meanwhile, as illustrated in FIG. 8B, in a case where the mirror 39 is used in the light reception system 23, light traveling across the anterior chamber 13 of the eyeball 10 passes through the cornea 14, is incident on the light reception system 23, and is reflected by the mirror 29, thereby being incident on the compensator 31, the analyzer 33, and the light reception portion 35. Therefore, the light incident on the compensator 31 is light of which the polarization state is further changed due to reflection of the mirror 39.

The polarization state of light emitted from the eyeball is influenced by double refraction of the cornea (cornea double refraction). In addition, the angle of light emitted from the eyeball is influenced by the shape and the like of the cornea. Here, the cornea double refraction depends on constitutive substances and the shape of the cornea and varies due to an individual difference, a daily variation, a subtle ocular movement, and the like. In addition, the shape of the cornea also varies due to an individual difference and a daily variation. Therefore, when the mirror 39 is used in the light reception system 23, rays of light in polarization states influenced by the cornea double refraction are incident on the mirror 39 at various angles. When the rays of incident light are reflected by the mirror 39 of the light reception system 23, the polarization states further change so as to be different from each other for each of the polarization states and the angles of the rays of incident light. That is, the light reception portion 35 outputs a composite signal of a change of the polarization state caused by light traveling across the anterior chamber 13, and a change of the polarization state caused by the mirror 39.

Therefore, similar to the mirror 39 of the light emission system 21, even if the refractive index and the incident angle of the mirror 29 are known, it is difficult to extract a signal of the former from the composite signal of a change of the polarization state caused by light traveling across the anterior chamber 13, and a change of the polarization state caused by the mirror 39. Thus, the accuracy of optical measurement related to the aqueous humor deteriorates.

As described above, in the configuration of the optical measurement apparatus 1 for an eyeball, in which the first exemplary embodiment is applied, the light emission system 21 is disposed on the inner side (inner canthus side) having an insufficient space. The optical path 28 is refracted by providing the mirror 29. The light reception system 23 is disposed on the outer side (outer canthus side) having a space. The mirror is not used (not involved). In consideration of a change of the polarization state caused by the mirror 29, the optical rotation degree α_{M} of the optically active substance contained in the aqueous humor is more accurately calculated.

### Second Exemplary Embodiment

According to the optical measurement apparatus 1 for an eyeball, in which the first exemplary embodiment is applied, in the light emission system 21 of the optical system 20, the light emission portion 25 and the polarizer 27 are fixed. The optical path 28 is set to pass through the anterior chamber 13 so as to travel across the anterior chamber 13 and to be incident on the light reception system 23 by changing the angle of the mirror 29.

According to an optical measurement apparatus 1 for an eyeball, in which a second exemplary embodiment is applied, in the light emission system 21 of the optical system 20, the light emission portion 25, the polarizer 27, and the mirror 29 are fixed to a fixing member 38. The angle of the light emission system 21 in its entirety is changed by the fixing member 38, and the optical path 28 is set to pass through the anterior chamber 13 so as to travel across the anterior chamber 13 and to be incident on the light reception system 23.

Similar to the first exemplary embodiment, the light reception system 23 is configured not to use (not to involve) the mirror.

In the optical measurement apparatus 1 for an eyeball, in which the second exemplary embodiment is applied, the light emission system 21 in the optical system 20 is different from that of the optical measurement apparatus 1 for an eyeball, in which the first exemplary embodiment is applied. However, other configurations are the same. Thus, in the following, the light emission system 21 in the optical system 20 of the optical measurement apparatus 1 for an eyeball, in which the second exemplary embodiment is applied, will be described.

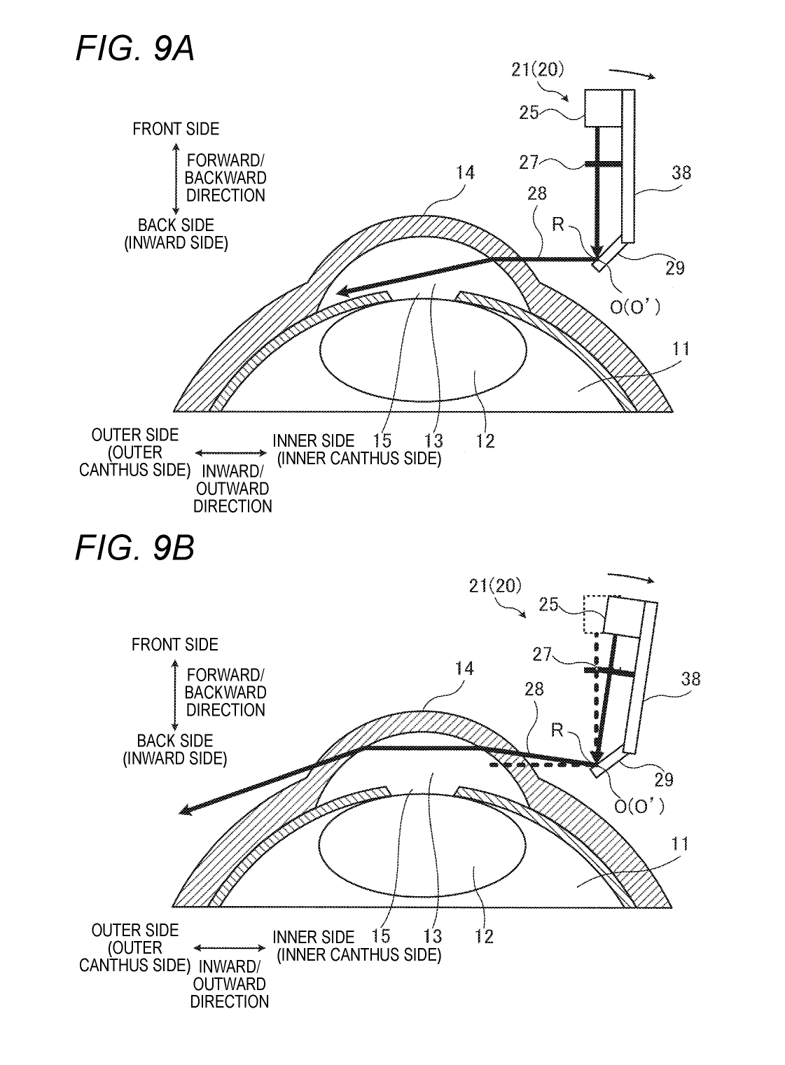

FIGS. 9A and 9B are views describing the light emission system 21 in the optical system 20 of the optical measurement apparatus 1 for an eyeball, in which the second exemplary embodiment is applied. Here, FIG. 9A illustrates a case where the optical path 28 does not pass through the anterior chamber 13 so as to travel across the anterior chamber 13, and FIG. 9B illustrates a case where the optical path 28 passes through the anterior chamber 13 so as to travel across the anterior chamber 13.

As illustrated in FIG. 9A, in the light emission system 21 in the optical system 20, the light emission portion 25, the polarizer 27, and the mirror 29 are fixed to the fixing member 38. The angle of the mirror 29 is also fixed by the fixing member 38. That is, the angle of the mirror 29 cannot be independently changed with respect to the light emission portion 25.

As illustrated in FIG. 9B, the fixing member 38 as a whole including the light emission portion 25, the polarizer 27, and the mirror 29 is rotated around the axis O-O′. Accordingly, the optical path 28 is set so as to pass through the anterior chamber 13 and to travel across the anterior chamber 13.

The position of the axis O-O′ may be provided on a side to the light emission portion 25 closer than the center of the light emission system 21 in its entirety in the length direction. However, as described in the first exemplary embodiment, as the axis O-O′ and the reflection point R of the mirror 29 are separated from each other in distance, the possibility that the mirror 29 hits the face (eyeball 10) in a case where the mirror 29 is rotated increases. Thus, when the axis O-O′ is provided on a side to the mirror 29 closer than the center of the light emission system 21 in its entirety in the length direction, compared to a case of being provided on a side close to the light emission portion 25, the possibility that the mirror 29 hits the face (eyeball 10) is reduced. In addition, when the axis O-O′ is provided in the region where the mirror 29 is provided in the light emission system 21 in its entirety in the length direction, the possibility that the mirror 29 hits the face (eyeball 10) is further reduced. In FIGS. 9A and 9B, as described in the first exemplary embodiment, the axis O-O′ passes through the reflection point R of the mirror 29 and is provided near the face side.

As described above, the light emission system 21 in the optical system 20 of the optical measurement apparatus 1 for an eyeball, in which the second exemplary embodiment is applied, rotates integrally with respect to the axis O-O′ via the fixing member 38. Therefore, even if the light emission system 21 is rotated, the incident angle of light incident on the mirror 29 does not change. Thus, the polarization state of light reflected from the mirror 29 does not change.

Therefore, according to the optical measurement apparatus 1 for an eyeball, in which the second exemplary embodiment is applied, being different from the optical measurement apparatus 1 for an eyeball, in which the first exemplary embodiment is applied, there is no need to consider the polarization state of light reflected from the mirror 29 every time the angle of the mirror 29 is changed.

As described above, in the configuration of the optical measurement apparatus 1 for an eyeball, in which the second exemplary embodiment is applied, the light emission system 21 is disposed on the inner side (inner canthus side) having an insufficient space. The optical path 28 is refracted by providing the mirror 29. The light reception system 23 is disposed on the outer side (outer canthus side) having a space. The mirror is not used (not involved). Thus, the optical rotation degree α_{M} of the optically active substance contained in the aqueous humor is likely to be more accurately calculated. In addition, even if the light emission system 21 is rotated, the polarization state is not changed due to the mirror 29. Therefore, even in a case where the light emission system 21 is rotated, there is no need to consider plural polarization states different from each other.

### Third Exemplary Embodiment

According to the optical measurement apparatus 1 for an eyeball, in which the second exemplary embodiment is applied, the light emission system 21 of the optical system 20 is moved around the axis O-O′ supported by the support portions 50B and 50C, and the optical path 28 is set to pass through the anterior chamber 13 so as to travel across the anterior chamber 13 and to be incident on the light reception system 23.

According to the optical measurement apparatus 1 for an eyeball, in which a third exemplary embodiment is applied, instead of the support portions 50B and 50C, a rail 51 is used. The light emission system 21 is moved on the rail 51, and the optical path 28 is set to pass through the anterior chamber 13 so as to travel across the anterior chamber 13 and to be incident on the light reception system 23.

Similar to the first exemplary embodiment, the light reception system 23 is configured not to use (not to involve) the mirror.

According to the optical measurement apparatus 1 for an eyeball, in which the third exemplary embodiment is applied, the light emission system 21 in the optical system 20 is different from that of the optical measurement apparatus 1 for an eyeball, in which the second exemplary embodiment is applied. However, other configurations are the same, Thus, in the following, the light emission system 21 in the optical system 20 of the optical measurement apparatus 1 for an eyeball, in which the third exemplary embodiment is applied, will be described.

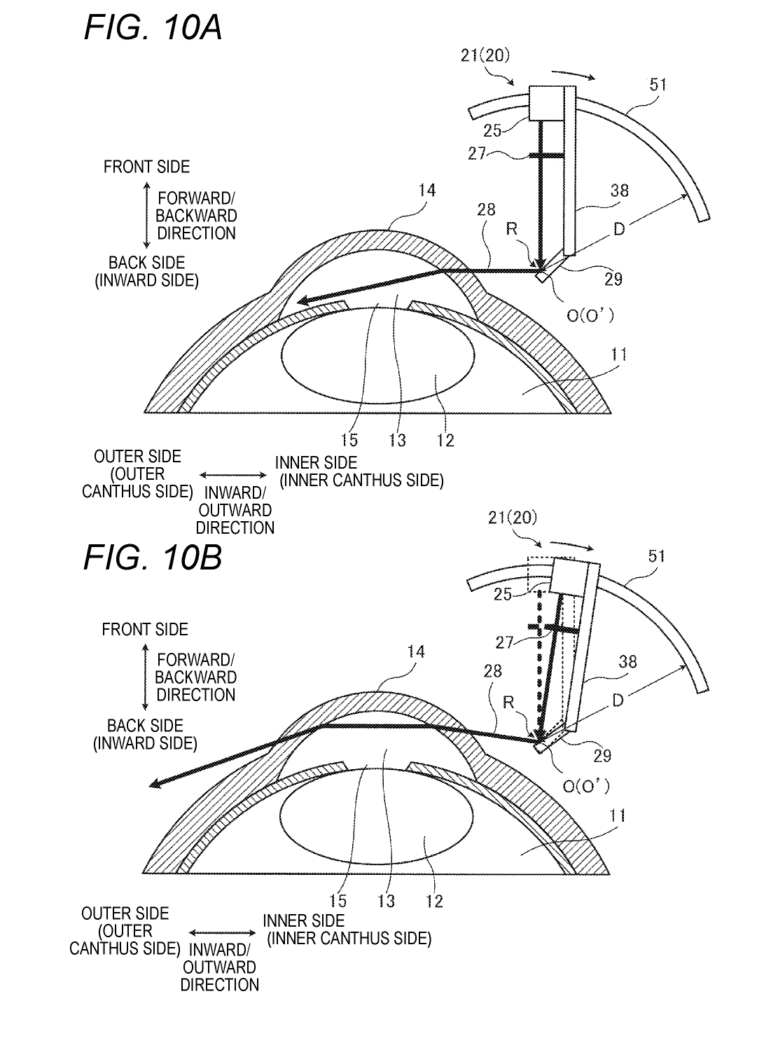

FIGS. 10A and 10B are views describing the light emission system 21 in the optical system 20 of the optical measurement apparatus 1 for an eyeball, in which the third exemplary embodiment is applied. Here, FIG. 10A illustrates a case where the optical path 28 does not pass through the anterior chamber 13 so as to travel across the anterior chamber 13, and FIG. 10B illustrates a case where the optical path 28 passes through the anterior chamber 13 so as to travel across the anterior chamber 13.

As illustrated in FIG. 10A, similar to the second exemplary embodiment, the light emission system 21 in the optical system 20 includes the fixing member 38 in addition to the light emission portion 25, the polarizer 27, and the mirror 29. The light emission portion 25, the polarizer 27, and the mirror 29 are fixed to the fixing member 38. Moreover, the angle of the mirror 29 is also fixed by the fixing member 38. That is, the angle of the mirror 29 cannot be independently changed with respect to the light emission portion 25.

The light emission system 21 is set such that the light emission portion 25 side moves on the rail 51 having a radius D. For example, the rail 51 is fixed to the cylindrical main body 50A of the holding section 50. The radius D of the rail 51 is set while having the reflection point R of the optical path 28 on the mirror 29 as the center. Thus, even if the light emission system 21 is moved on the rail 51, the reflection point R does not move.

The light emission system 21 may be manually moved on the rail 51 by the measurement subject. In this case, the rail 51 is another example of the adjustment section. In addition, a motor or the like may be included in a portion where the light emission system 21 is supported by the rail 51. A rotary axis of the motor and the surface of the rail 51 may come into contact with each other and the light emission system 21 may be moved by rotating the motor based on the controlling of the control section 40. In this case, a mechanism of moving the rail 51 and the light emission system 21 on the rail 51 is further another example of the adjustment section.

Therefore, being different from the optical measurement apparatus 1 for an eyeball, in which the first exemplary embodiment is applied, there is no need to consider the polarization state of light reflected from the mirror 29 every time the angle of the mirror 29 is changed.

As described above, in the configuration of the optical measurement apparatus 1 for an eyeball, in which the third exemplary embodiment is applied, the light emission system 21 is disposed on the inner side (inner canthus side) having an insufficient space. The optical path 28 is refracted by providing the mirror 29. The light reception system 23 is disposed on the outer side (outer canthus side) having a space. The mirror is not used (not involved). Thus, the optical rotation degree α_{M} of the optically active substance contained in the aqueous humor is likely to be more accurately calculated. In addition, even if the light emission system 21 is rotated, the polarization state is not changed due to the mirror 29. Therefore, even in a case where the light emission system 21 is rotated, there is no need to consider plural polarization states different from each other.

Hereinbefore various exemplary embodiments have been described. However, the exemplary embodiments may be configured in a combination.

In addition, the present disclosure is not limited to any of the exemplary embodiments described above and can be executed in various forms without departing from the gist of the present disclosure.

What is claimed is:

1. An optical measurement apparatus for an eyeball, comprising:
a light irradiation section that is disposed on an inner canthus side of the eyeball and that includes
a light source,
a polarization portion that performs polarization control of light from the light source, and
a light reflection portion that is disposed closer to an inward side of an eyeball than the polarization portion is and that reflects light subjected to the polarization control by the polarization portion such that the light travels across an anterior chamber of the eyeball; and
a light reception section that is disposed on an outer canthus side of the eyeball, that receives the light traveling across the anterior chamber without further going through a light reflection portion that changes a polarization state of light, and that detects a change of the polarization state of the received light.

2. The optical measurement apparatus for an eyeball, according to claim 1, further comprising
an adjustment section that adjusts a direction of light reflected from the light reflection portion in the light irradiation section toward the anterior chamber such that the reflected light travels across the anterior chamber and is received by the light reception section and
an angle measurement section that measures an incident angle of the light subjected to the polarization control and incident on the light reflection portion in the light irradiation section, wherein
the adjustment section adjusts an angle of the light reflection portion in the light irradiation section with respect to the light source.

3. The optical measurement apparatus for an eyeball, according to claim 2, wherein
the adjustment section adjusts the angle of the light reflection portion with respect to the light source by rotating the light reflection portion around an axis provided in the light reflection portion in the light irradiation section.

4. The optical measurement apparatus for an eyeball, according to claim 1, wherein
the light irradiation section further includes a fixing member that fixes a positional relationship among the light source, the polarization portion, and the light reflection portion, and
the fixing member fixes an angle of the light reflection portion with respect to the light source.

5. The optical measurement apparatus for an eyeball, according to claim 4, further comprising:
an adjustment section that adjusts a direction of light subjected to the polarization control and reflected from the light reflection portion in the light irradiation section by rotating the fixing member in the light irradiation section, wherein
the adjustment section rotates the fixing member around an axis provided in the light reflection portion that is fixed to the fixing member.
